(19) United States
(12) Patent Application Publication
(10) Pub. No.: US 2006/0093616 A1
(43) Pub. Date: May 4, 2006

(54) Process for vaccinating eucaryotic hosts and for protecting against SARS-CoV infection

(76) Inventors: Ralf Altmeyer, Hong Kong (HK); Beatrice Nal-Rogier, Mid-levels (HK); Cheman Chan, North Point (HK); Yiu Wing Kam, Ap Lei Chau (HK); Francois Kien, Causeway (HK); Lewis Siu, Chaiwan (HK); Jane Tse, Hong Kong (HK); Isabelle Staropoli, Paris (FR); J. Claude Manugurrea, Paris (FR); Malik Peiris, Pokfulam (HK)

Correspondence Address: FINNEGAN, HENDERSON, FARABOW,;GARRETT & DUNNER, L.L.P., 901 New York Avenue, NW, Washington, DC, 20001-4413, US

(21) Appl. No.: 11/236,856

(22) Filed: Sep. 28, 2005

Related U.S. Application Data
(60) Provisional application No. 60/614,027, filed on Sep. 29, 2004.

(51) Int. Cl. A61K 39/12; A61K 39/215; C12Q 1/70; C07K 16/10

(52) U.S. Cl. 424/186.1; 424/221.1; 435/5; 530/388.3

(57) ABSTRACT

The present invention relates to a process for vaccinating humans and for protecting against SARS-CoV infection.

### CROSS-REFERENCE TO RELATED APPLICATIONS

This application is based on and claims the benefit of U.S. Provisional Application No. 60/614,027, filed Sep. 29, 2004, (Attorney Docket No. 3495.6106). The entire disclosure of this application is relied upon and incorporated by reference herein.

### FIELD OF THE INVENTION

The present invention relates to a process for vaccinating eukaryotic hosts and particularly humans and for protecting against SARS-CoV infection using trimeric S-proteins of SARS-CoV. This invention is also directed to purified and isolated antibodies generated against these proteins and their complex, and the use of such antibodies and proteins in diagnostic methods, kits, vaccines, or antiviral therapy.

### BACKGROUND OF THE INVENTION

Severe acute respiratory syndrome (SARS) is an emerging disease caused by a novel coronavirus, SARS-CoV, which infected more than 8000 people and caused 774 deaths worldwide since November 2002 (Peiris et al., 2003). Convalescent patients have high-titer neutralizing antibodies (nAb) while patients developing severe forms of the disease show a decrease in antibody titer as the disease progresses. At present, there is neither a vaccine nor a specific anti-viral treatment available. Antibody transfer experiments indicate that the humoral neutralizing antibody response alone can protect against SARS-CoV (Subbarao et al., 2004; Yang et al., 2004b). The receptor binding protein S, or Spike, is the key target of the neutralizing response, demonstrated by protection through passive transfer of S-protein specific sera in naïve mice (Bisht et al., 2004; Yang et al., 2004b) or ferrets (ter Meulen et al., 2004). The S protein is a 150 to 180 kDa highly glycosylated trimeric class-I fusion protein (Bosch et al., 2003; Song et al., 2004) responsible for receptor binding and virus-membrane fusion and tissue tropism of coronaviruses. While DC-SIGN on dendritic cells binds SARS-CoV S-protein, this interaction does not lead to virus-cell fusion and productive replication (Yang et al., 2004a). Angiotensin converting enzyme 2 (ACE2) has been identified as the receptor for the virus entry into susceptible target cells (Li et al., 2003).

Immunization with gene or viral vectors encoding fragments or full-length S-proteins induce SARS-CoV nAb (Sui et al., 2004; Zeng et al., 2004; Zhang et al., 2004) and protection (Buchholz et al., 2004; Bukreyev et al., 2004; Yang et al., 2004b). Both the putative S1 (Sui et al., 2004; Zeng et al., 2004) and S2 subunits (Zeng et al., 2004; Zhang et al., 2004) of S are immunogenic. These recent data for SARS-CoV are corroborated by earlier findings for other coronaviruses such as Mouse Hepatitis Virus (Daniel and Talbot, 1990), Avian Infectious Bronchitis Virus (Ignjatovic and Galli, 1994), Transmissible Gastroenteritis Virus (Torres et al., 1995) and Infectious Bronchitis Virus (Song et al., 1998). Several vaccine approaches have been described for SARS, including whole inactivated virus (WIV) (Takasuka et al., 2004), DNA (Yang et al., 2004b; Zeng et al., 2004) and viral vectors (Bisht et al., 2004; Bukreyev et al., 2004; Gao et al., 2003). Although such vaccines induce a specific, neutralizing immune response there are safety concerns with respect to use in humans.

There is a considerable need for the development of a detailed understanding of SARS-CoV proteins, which should clarify the mechanisms by which SARS-CoV induces infection. Such an understanding can lead to effective means to treat or control the infection, as well as aid in the diagnosis of SARS-CoV infection in humans.

### SUMMARY OF THE INVENTION

Accordingly, this invention aids in fulfilling these needs in the art. An aim of the present invention is to provide a composition containing a TriSpike protein inducing neutralizing antibodies in vivo and a process for vaccinating eukaryotic hosts as humans against SARS-CoV infection and/or diseases induced by SARS-CoV. The present invention concerns more particularly the administration of trimeric S-protein (TriSpike) of SARS-CoV to a host with an acceptable physiological carrier and/or an adjuvant.

Purified polyclonal or monoclonal antibodies that bind to trimeric S-protein (TriSpike) are encompassed by the invention.

Immunological complexes between the trimeric S-protein (TriSpike) and antibodies or serum containing neutralizing antibodies of the invention recognizing the proteins are also provided. The immunological complexes can be labeled with an immunoassay label selected from the group consisting of radioactive, enzymatic, fluorescent, chemiluminescent labels, and chromophores.

Furthermore, this invention provides a method for detecting infection by SARS-CoV. The method comprises providing a composition comprising a biological material suspected of being infected with SARS-CoV, and assaying for the presence of trimeric S-protein (TriSpike) of SARS-CoV. The proteins are typically assayed by electrophoresis or by immunoassay with antibodies of the invention that are immunologically reactive with trimeric S-protein (TriSpike).

This invention also provides an in vitro diagnostic method for the detection of the presence or absence of antigens comprising the trimeric S-protein (TriSpike), which bind to an antibody of the invention. The method comprises contacting the antigen with a biological fluid for a time and under conditions sufficient for the antibodies and the proteins in the biological fluid to form an antigen-antibody complex, and then detecting the formation of the complex. The detecting step can further comprising measuring the formation of the antigen-antibody complex. The formation of the antigen-antibody complex is preferably measured by immunoassay based on Western blot technique, ELISA (enzyme linked immunosorbent assay), indirect immunofluorescent assay, or immunoprecipitation assay.

A diagnostic kit for the detection of the presence or absence of the trimeric S-protein (TriSpike) antigen, contains antibodies of the invention, and means for detecting the formation of immune complex between the antigen and antibodies. The antibodies and the means are present in an amount sufficient to perform the detection.

This invention also provides an immunogenic composition comprising a trimeric S-protein (TriSpike) in an amount sufficient to induce an immunogenic or protective response in vivo, in association optionally with a pharmaceutically acceptable carrier therefor. A vaccine composition of the invention comprises the purified trimeric S-protein (TriSpike) capable to induce in vivo the production of neutralizing antibodies against a SARS-CoV virus and a pharmaceutically acceptable carrier therefor.

The antibodies of this invention are useful as a portion of a diagnostic composition for detecting the presence of antigenic proteins associated with SARS-CoV. The antibodies of the invention can be also employed to inactivate the virus, reduce the viability of the virus in vivo, or inhibit or prevent viral replication. The ability to elicit virus-neutralizing antibodies is especially important when the trimeric S-protein (TriSpike) is used in immunizing or vaccinating compositions.

The purified antibodies according to the invention can also be a reagent in a diagnostic process to quantify or identify in a serum of a patient the presence or absence of the SARS CoV virus or antibodies against this virus raised by the said patient.

### BRIEF DESCRIPTION OF THE DRAWINGS

This invention will be more fully described with reference to the drawings in which:

FIG. 1. Biochemical characterization of purified trimeric SARS-CoV S-protein (TriSpike). (A) S-protein was expressed in BHK-21 cells and purified by immunoaffinity as described in Examples 1 and 2. Eluted protein was treated as indicated and analyzed by SDS-Page and Western Blot using M2 mAb. (B) Recognition of TriSpike protein on Western Blot by human SARS patient sera. TriSpike was analyzed by SDS-PAGE under non-reducing condition blotted and reacted with convalescent SARS patient sera (lanes 3 to 7) and normal human sera (lanes 1, 2) at 1/500 dilution. Immune complexes were detected with HRP conjugated goat anti-human IgG polyclonal antibody.

FIG. 2. Immunogenicity of TriSpike. Sera from vaccinated and control mice were analyzed for reactivity with S-protein. (A-C) A high-titer neutralizing SARS patient serum, a rabbit serum against S1, and M2 monoclonal antibody against the FLAG peptide were used as controls. (A) Western Blot analysis of pooled sera from mice immunized with S-RNA (d0) and TriSpike (d14, d35). Sera were collected at indicated time points and used at 1/500 dilution for Western Blot analysis. All sera were reacted with FLAG-tagged control protein (BAP-FLAG) to assess antibody production against the FLAG tag. Immune complexes were detected with HRP-conjugated goat anti-mouse, human or rabbit IgG polyclonal antibody. (B) same as (A) except that Western Blot analysis was performed with pooled sera from mice immunized with TriSpike alone on day 0 (Groups A, B), 14 (Group B) and 41 (Groups A, B) and bled on indicated days. (C) Reactivity of immune sera with native S protein. Sera, diluted 1/100, from mice immunized with S-RNA plus TriSpike (upper panel) or TriSpike only (lower panel) were reacted with live BHK-21 cells expressing S-protein at the plasma membrane. Immune complexes and IgG isotypes were identified using FITC-conjugated goat anti-mouse IgG (H+ L) and rat-anti-mouse IgG1 or IgG2a antibodies.

FIG. 3. Sera from immunized mice react with SARS-CoV infected cells. (A) Sera collected at indicated times from S-RNA plus TriSpike immunized animals were pooled and reacted with SARS-CoV-infected FRhk-4 cells at 1/50 dilution prior to detection with TexasRed-conjugated goat anti-mouse IgG (H+ L) antibody and nuclear counterstaining with DAPI. (B) Sera collected at indicated times from groups A and B of TriSpike immunized animals were pooled and reacted with SARS-CoV13 infected Vero cells at 1/50 dilution prior to detection with TexasRed-conjugated goat anti-mouse IgG (H+ L) antibody.

FIG. 4. Sera from immunized mice inhibit S-protein binding to the ACE2 receptor. Soluble recombinant human ACE2 (sACE2) was incubated with recombinant SFLAG protein preadsorbed onto anti-FLAG M2 agarose affinity gel and preincubated with d42 neutralizing serum of S-RNA plus TriSpike immunized animals. M2 agarose affinity coated with BAP-FLAG protein was used as a negative control. S-protein-ACE2 complexes were washed, separated by SDS-PAGE and co-precipitated ACE2 detected by Western blot with a goat anti-ACE2 polyclonal antibody. Immune complexes were detected with a mouse HRP-conjugated anti-goat IgG monoclonal antibody.

### DETAILED DESCRIPTION OF THE INVENTION

It was reasoned that expression of a full-length S-protein would generate trimeric molecules with native antigenic and immunogenic properties mimicking the native S-protein on the virion surface. Biochemically purified or pure trimers should be able to induce a strong neutralizing response against the receptor binding domain of S-protein to prevent the initiation of an infectious cycle. It was discovered that trimeric S-protein alone is capable of inducing specific high-titer neutralizing antibodies in vivo, which inhibit virus attachment to the ACE2 entry receptor.

The term “purified” as used herein, means that the trimeric S-protein (TriSpike) is essentially free of association with other proteins or polypeptides, for example, as a purification product of recombinant host cell culture or as a purified product from a non-recombinant source. The term “substantially purified” as used herein, refers to a mixture that contains trimeric S-protein (TriSpike) and is essentially free of association with other proteins or polypeptides, but for the presence of known proteins that can be removed using a specific antibody. The substantially purified trimeric S-protein (TriSpike) can be used as antigens.

A trimeric S-protein (TriSpike) “variant” as referred to herein means a polypeptide substantially homologous to native trimeric S-protein of SARS-CoV, but which has an amino acid sequence different from that of native trimeric S-protein of SARS-CoV because of one or more deletions, insertions, or substitutions. The variant amino acid sequence preferably is at least 95% identical to a native trimeric S-protein of SARS-CoV amino acid sequence, most preferably at least 98% identical. The percent identity can be determined, for example by comparing sequence information using the GAP computer program, version 6.0 described by Devereux et al. (Nucl. Acids Res. 12:387, 1984) and available from the University of Wisconsin Genetics Computer Group (UWGCG). The GAP program utilizes the alignment method of Needleman and Wunsch (J. Mol. Biol. 48:443, 1970), as revised by Smith and Waterman (Adv. Appl. Math 2:482, 1981). The preferred default parameters for the GAP program include: (1) a unary comparison matrix (containing a value of 1 for identities and 0 for non-identities) for nucleotides, and the weighted comparison matrix of Gribskov and Burgess, Nucl. Acids Res. 14:6745, 1986, as described by Schwartz and Dayhoff, eds., Atlas of Protein Sequence and Structure, National Biomedical Research Foundation, pp. 353-358, 1979; (2) a penalty of 3.0 for each gap and an additional 0.10 penalty for each symbol in each gap; and (3) no penalty for end gaps.

Variants can comprise conservatively substituted sequences, meaning that a given amino acid residue is replaced by a residue having similar physiochemical characteristics. Examples of conservative substitutions include substitution of one aliphatic residue for another, such as Ile, Val, Leu, or Ala for one another, or substitutions of one polar residue for another, such as between Lys and Arg; Glu and Asp; or Gln and Asn. Other such conservative substitutions, for example, substitutions of entire regions having similar hydrophobicity characteristics, are well known. The use of naturally occurring trimeric S-protein of SARS-CoV variants are also encompassed by the invention. Examples of such variants are proteins that result from alternate mRNA splicing events or from proteolytic cleavage of the trimeric S-protein of SARS-CoV. Variations attributable to proteolysis include, for example, differences in the termini upon expression in different types of host cells, due to proteolytic removal of one or more terminal amino acids from the trimeric S-protein of SARS-CoV. Variations attributable to frameshifting include, for example, differences in the termini upon expression in different types of host cells due to different amino acids.

As stated above, the invention utilizes isolated and purified, or homogeneous, trimeric S-protein (TriSpike), both recombinant and non-recombinant. Variants and derivatives of native trimeric S-protein of SARS-CoV that can be used as antigens can be obtained by mutations of nucleotide sequences coding for native trimeric S-protein of SARS-CoV. Alterations of the native amino acid sequence can be accomplished by any of a number of conventional methods. Mutations can be introduced at particular loci by synthesizing oligonucleotides containing a mutant sequence, flanked by restriction sites enabling ligation to fragments of the native sequence. Following ligation, the resulting reconstructed sequence encodes an analog having the desired amino acid insertion, substitution, or deletion. Alternatively, oligonucleotide-directed site-specific mutagenesis procedures can be employed to provide an altered gene wherein predetermined codons can be altered by substitution, deletion, or insertion.

Within an aspect of the invention, native or recombinant trimeric S-protein (TriSpike) can be utilized to prepare antibodies that specifically bind to native or recombinant trimeric S-protein (TriSpike). The term “antibodies” is meant to include polyclonal antibodies, monoclonal antibodies, fragments thereof such as F(ab′)2 and Fab fragments, as well as any recombinantly produced binding partners. Antibodies are defined to be specifically binding if they bind to the trimeric S-protein (TriSpike) with a Kₐ of greater than or equal to about 10⁷ M⁻¹. Affinities of binding partners or antibodies can be readily determined using conventional techniques, for example, those described by Scatchard et al., Ann. N.Y Acad. Sci., 51:660 (1949). Polyclonal antibodies can be readily generated from a variety of sources, for example, horses, cows, goats, sheep, dogs, chickens, rabbits, mice, or rats, using procedures that are well known in the art.

It will be understood that the present invention is intended to encompass use of the previously described proteins in isolated or purified form, whether obtained using the techniques described herein or other methods. In a preferred embodiment of this invention, the trimeric S-protein (TriSpike) is substantially free of human tissue and human tissue components, nucleic acids, extraneous proteins and lipids, and adventitious microorganisms, such as bacteria and viruses. It will also be understood that the invention encompasses the use of equivalent proteins having substantially the same biological and immunogenic properties. Thus, this invention is intended to cover the use of serotypic variants of the proteins.

Once the native or recombinant trimeric S-protein (TriSpike) has been obtained, it can be used to produce polyclonal and monoclonal antibodies reactive therewith. Thus, the protein can be used to immunize an animal host by techniques known in the art. Such techniques usually involve inoculation, but they may involve other modes of administration. A sufficient amount of the protein or the polypeptide is administered to create an immunogenic response in the animal host. Any host that produces antibodies to the antigen (protein) can be used. Once the animal has been immunized and sufficient time has passed for it to begin producing antibodies to the antigen, polyclonal antibodies can be recovered. The general method comprises removing blood from the animal and separating the serum from the blood. The serum, which contains antibodies to the antigen, can be used as an antiserum to the antigen. Alternatively, the antibodies can be recovered from the serum. Affinity purification is a preferred technique for recovering purified polyclonal antibodies to the antigen from the serum.

Monoclonal antibodies to the native or recombinant S-protein (TriSpike) can also be prepared. One method for producing monoclonal antibodies reactive with the protein comprises the steps of immunizing a host with the protein; recovering antibody producing cells from the spleen of the host; fusing the antibody producing cells with myeloma cells deficient in the enzyme hypoxanthine-guanine phosphoribosyl transferase to form hybridomas; selecting at least one of the hybridomas by growth in a medium comprising hypoxanthine, aminopterin, and thymidine; identifying at least one of the hybridomas that produces an antibody to the protein, culturing the identified hybridoma to produce antibody in a recoverable quantity; and recovering the antibodies produced by the cultured hybridoma.

These polyclonal or monoclonal antibodies can be used in a variety of applications. Among these is the neutralization of corresponding proteins or virus containing such proteins. They can also be used to detect viral antigens in biological preparations or in purifying corresponding proteins, glycoproteins, or mixtures thereof, for example when used in a affinity chromatographic columns.

The antibodies to trimeric S-protein (TriSpike) can be used to identify the S-protein of SARS-CoV in materials and to determine the concentration of the protein in those materials. Thus, the antibodies can be used for qualitative or quantitative determination of the virus in a material. Such materials of course include human tissue and human cells, as well as biological fluids, such as human body fluids, including human sera. When used as a reagent in an immunoassay for determining the presence or concentration of the protein of SARS-CoV, the antibodies of the present invention provide an assay that is convenient, rapid, sensitive, and specific.

More particularly, the antibodies of the invention can be employed for the detection of SARS-CoV by means of immunoassays that are well known for use in detecting or quantifying humoral components in fluids. Thus, antigen-antibody interactions can be directly observed or determined by secondary reactions, such as precipitation or agglutination. In addition, immunoelectrophoresis techniques can also be employed. For example, the classic combination of electrophoresis in agar followed by reaction with anti-serum can be utilized, as well as two-dimensional electrophoresis, rocket electrophoresis, and immunolabeling of polyacrylamide gel patterns (Western Blot or immunoblot). Other immunoassays in which the antibodies of the present invention can be employed include, but are not limited to, radioimmunoassay, competitive immunoprecipitation assay, enzyme immunoassay, and immunofluorescence assay. It will be understood that turbidimetric, colorimetric, and nephelometric techniques can be employed. An immunoassay based on Western Blot technique is preferred.

Immunoassays can be carried out by immobilizing one of the immunoreagents, either an antigen or an antibody to the antigen, on a carrier surface while retaining immunoreactivity of the reagent. The reciprocal immunoreagent can be unlabeled or labeled in such a manner that immunoreactivity is also retained. These techniques are especially suitable for use in enzyme immunoassays, such as enzyme linked immunosorbent assay (ELISA) and competitive inhibition enzyme immunoassay (CIEIA).

When either the antigen or antibody to the antigen is attached to a solid support, the support is usually a glass or plastic material. Plastic materials molded in the form of plates, tubes, beads, or disks are preferred. Examples of suitable plastic materials are polystyrene and polyvinyl chloride. If the immunoreagent does not readily bind to the solid support, a carrier material can be interposed between the reagent and the support. Examples of suitable carrier materials are proteins, such as bovine serum albumin, or chemical reagents, such as gluteraldehyde or urea. Coating of the solid phase can be carried out using conventional techniques.

The invention provides immunogenic trimeric S-protein (TriSpike), and more particularly, protective polypeptides for use in the preparation of immunogenic and vaccine compositions against SARS-CoV. These proteins and peptides can thus be employed as viral vaccines by administering the proteins and polypeptides to a mammal, such as a human, susceptible to SARS-CoV infection. Conventional modes of administration can be employed. For example, administration can be carried out by oral, respiratory, inhalation, or parenteral routes. Intradermal, subcutaneous, and intramuscular routes of administration are preferred when the vaccine is administered parenterally.

The ability of the trimeric S-protein (TriSpike) and vaccines of the invention to induce protective levels of neutralizing antibody in a host can be enhanced by emulsification with an adjuvant, incorporating in a liposome, coupling to a suitable carrier, or by combinations of these techniques. For example, the trimeric S-protein (TriSpike) can be administered with a conventional adjuvant, such as aluminum phosphate and aluminum hydroxide gel, in an amount sufficient to potentiate humoral or cell-mediated immune response in the host. Similarly, the trimeric S-protein (TriSpike) can be bound to lipid membranes or incorporated in lipid membranes to form liposomes. The use of nonpyrogenic lipids free of nucleic acids and other extraneous matter can be employed for this purpose. This invention also encompasses the use of subunit vaccines containing the protein.

The immunization schedule will depend upon several factors, such as the susceptibility of the host to infection and the age of the host. A single dose of the vaccine of the invention can be administered to the host or a primary course of immunization can be followed in which several doses at intervals of time are administered. Subsequent doses used as boosters can be administered as need following the primary course.

The trimeric S-protein (TriSpike) can be administered to the host in an amount sufficient to prevent or inhibit SARS-CoV infection or replication in vivo. In any event, the amount administered should be at least sufficient to protect the host against substantial immunosuppression, even though SARS-CoV infection may not be entirely prevented. An immunogenic response can be obtained by administering the trimeric S-protein (TriSpike) to the host in an amount of about 10 to about 500 micrograms protein per kilogram of body weight, preferably about 50 to about 100 micrograms protein per kilogram of body weight. The vaccines of the invention can be administered together with a physiologically acceptable carrier. For example, a diluent, such as water or a saline solution, can be employed.

Another aspect of the invention provides a method of DNA vaccination. The method also includes administering any combination of the nucleic acids encoding trimeric S-protein (TriSpike), the proteins and polypeptides per se, with or without carrier molecules, to an individual. In embodiments, the individual is an animal, and is preferably a mammal. More preferably, the mammal is selected from the group consisting of a human, a dog, a cat, a bovine, a pig, and a horse. In an especially preferred embodiment, the mammal is a human.

The methods of treating include administering immunogenic compositions comprising trimeric S-protein (TriSpike), but compositions comprising nucleic acids encoding trimeric S-protein (TriSpike) or a fragment thereof as well. Those of skill in the art are cognizant of the concept, application, and effectiveness of nucleic acid vaccines (e.g., DNA vaccines) and nucleic acid vaccine technology as well as protein and polypeptide based technologies. The nucleic acid based technology allows the administration of nucleic acids encoding trimeric S-protein (TriSpike), naked or encapsulated, directly to tissues and cells without the need for production of encoded proteins prior to administration. The technology is based on the ability of these nucleic acids to be taken up by cells of the recipient organism and expressed to produce an immunogenic determinant to which the recipient's immune system responds. Typically, the expressed antigens are displayed on the surface of cells that have taken up and expressed the nucleic acids, but expression and export of the encoded antigens into the circulatory system of the recipient individual is also within the scope of the present invention. Such nucleic acid vaccine technology includes, but is not limited to, delivery of naked DNA and RNA and delivery of expression vectors encoding trimeric S-protein (TriSpike).

Although it is within the present invention to deliver nucleic acids encoding trimeric S-protein (TriSpike) and carrier molecules as naked nucleic acid, the present invention also encompasses delivery of nucleic acids as part of larger or more complex compositions. Included among these delivery systems are viruses, virus-like particles, or bacteria containing the nucleic acid encoding trimeric S-protein (TriSpike). Also, complexes of nucleic acids and carrier molecules with cell permeabilizing compounds, such as liposomes, are included within the scope of the invention. Other compounds, such as molecular vectors (EP 696,191, Samain et al.) and delivery systems for nucleic acid vaccines are known to the skilled artisan and exemplified in, for example, WO 93 06223 and WO 90 11092, U.S. Pat. No. 5,580,859, and U.S. Pat. No. 5,589,466 (Vical's patents), which are incorporated by reference herein, and can be made and used without undue or excessive experimentation.

Although the compositions containing trimeric S-protein (TriSpike) or nucleic acids encoding it are termed “vaccine”, which provides a neutralizing or protective immune response, it is equally applicable to immunogenic compositions that do not result in a protective immune response. Such non-protection inducing, immunogenic compositions and methods are encompassed within the present invention.

To further achieve the objects and in accordance with the purposes of the present invention, a kit capable of diagnosing an SARS-CoV infection is described. This kit, in one embodiment, contains the antibodies of this invention.

Production of Immunopurified Trimeric S-Protein with Native Antigenicity.

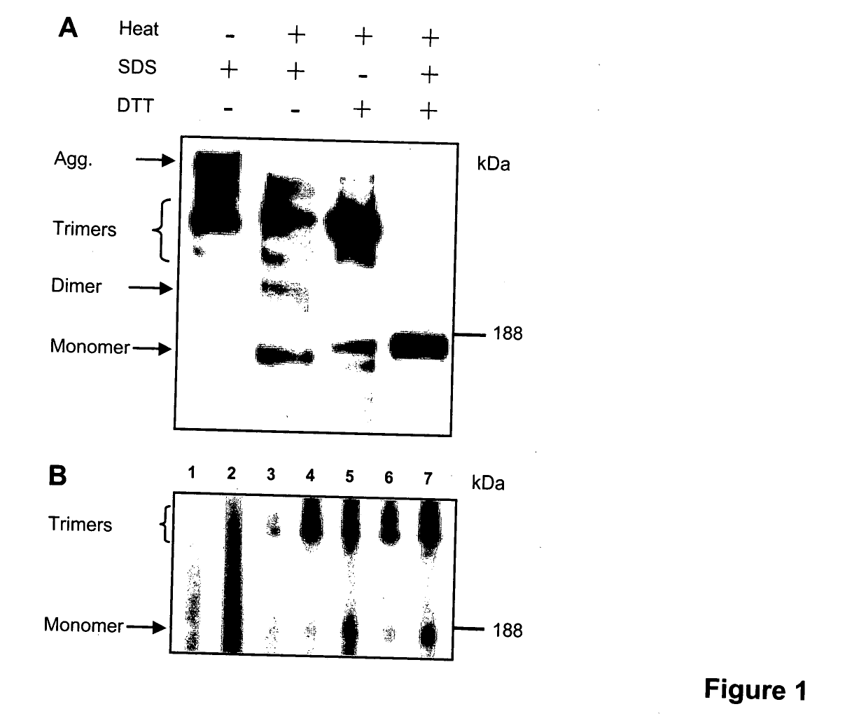

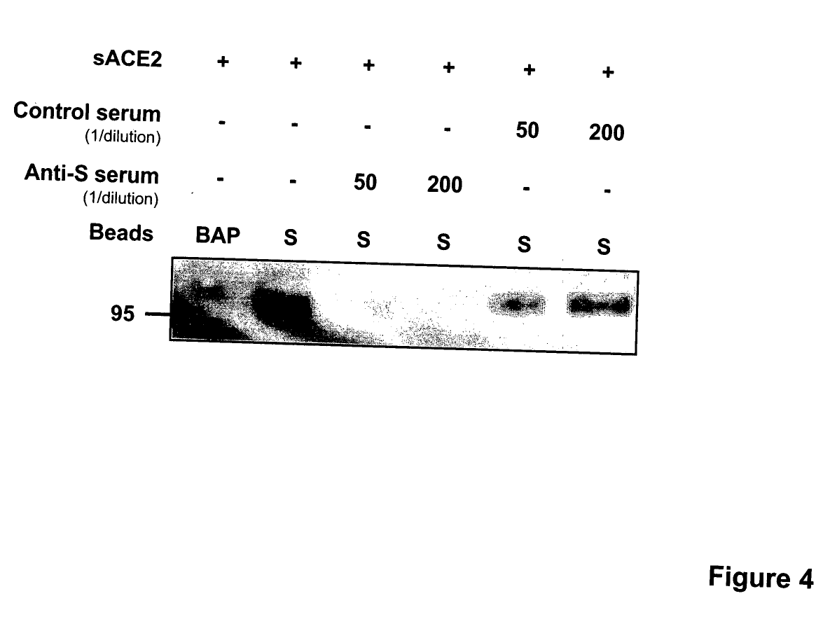

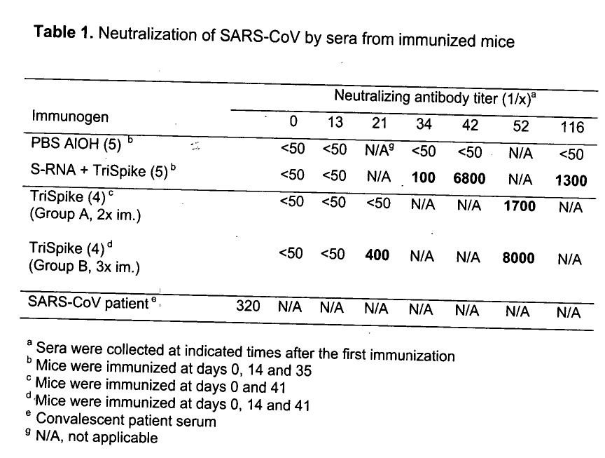

The defective Semliki Forest Virus vector coding for a full-length, codon optimized SARS-CoV S-protein fused to a C-terminal FLAG peptide was used. Trimeric S-protein (TriSpike) was purified by immunoaffinity from transfected or infected hamster cells (BHK-21). The overall yield of S-protein in this system is on the average 3 μg of immunopurified S-protein per 106 cells. Analysis of the apparent molecular weight of the protein by SDS-PAGE and Western Blot under non-reducing conditions revealed the predominant trimeric nature of the antigen (FIG. 1 A, lane 1). Higher molecular weight aggregates were occasionaly observed when the protein was not heat denatured prior to SDS-PAGE. Trimers dissociate partly into monomers when the protein is heat-denatured in the presence of SDS (FIG. 1 A, lane 2), but not if trimers are treated with DTT without SDS indicating that disulfide bonds are burried within the S monomer and trimer and not accessible to the reducing agent (FIG. 1 A, lane 3). As expected, trimers dissociate completely into monomers when heat-denatured in SDS and DTT (FIG. 1 A, lane 4). The trimeric and monomeric S-protein frequently migrate as doublets (FIG. 1 A) which represent high-mannose glycoforms from proteins that reside in the ER at the time of lysis and glycoforms from proteins that have acquired complex N-glycans in the median-Golgi (NaI, Chan et al., unpublished observations). Purified trimeric S-protein, termed TriSpike throughout this invention, has native antigenicity shown by reactivity with sera from 5 convalescent SARS patients by Western Blot (FIG. 1 B) and 11 sera tested by FACS (data not shown). The native fold was further underscored by the specific binding of the TriSpike protein with soluble ACE2 receptor (FIG. 4, lanes 1 and 2). These results strongly argue that purified TriSpike molecules mimick the native trimeric S-protein on the virion surface. Beyond its use as a vaccine, TriSpike will be an interesting tool for the development of sensitive and specific SARS serodiagnostic assays.

TriSpike Induces High-Titer Antibodies Against SARS-CoV S-Protein.

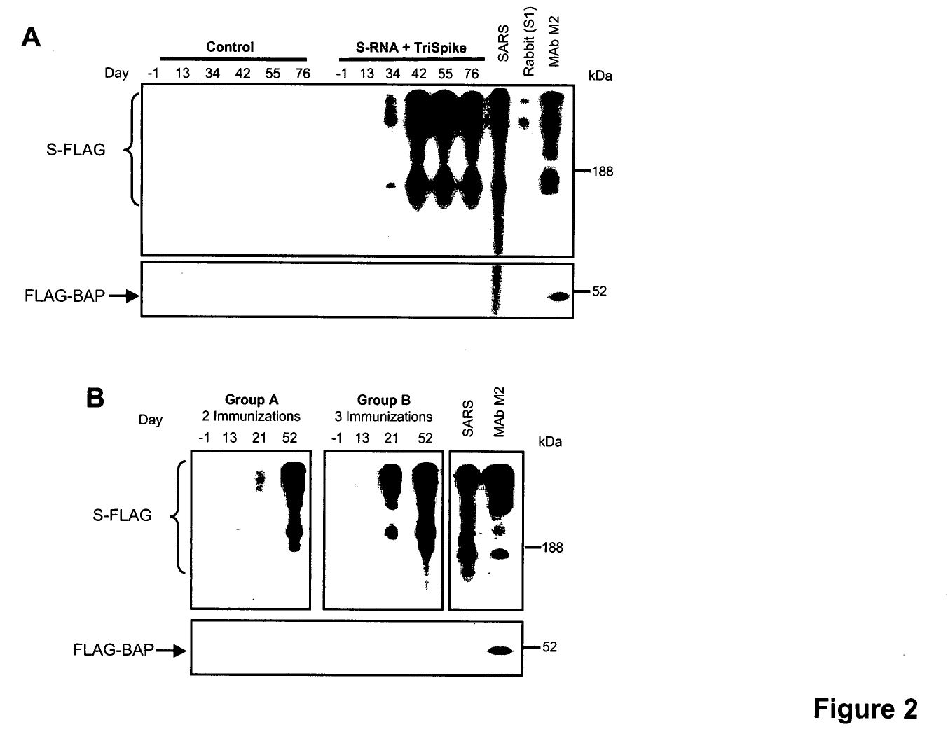

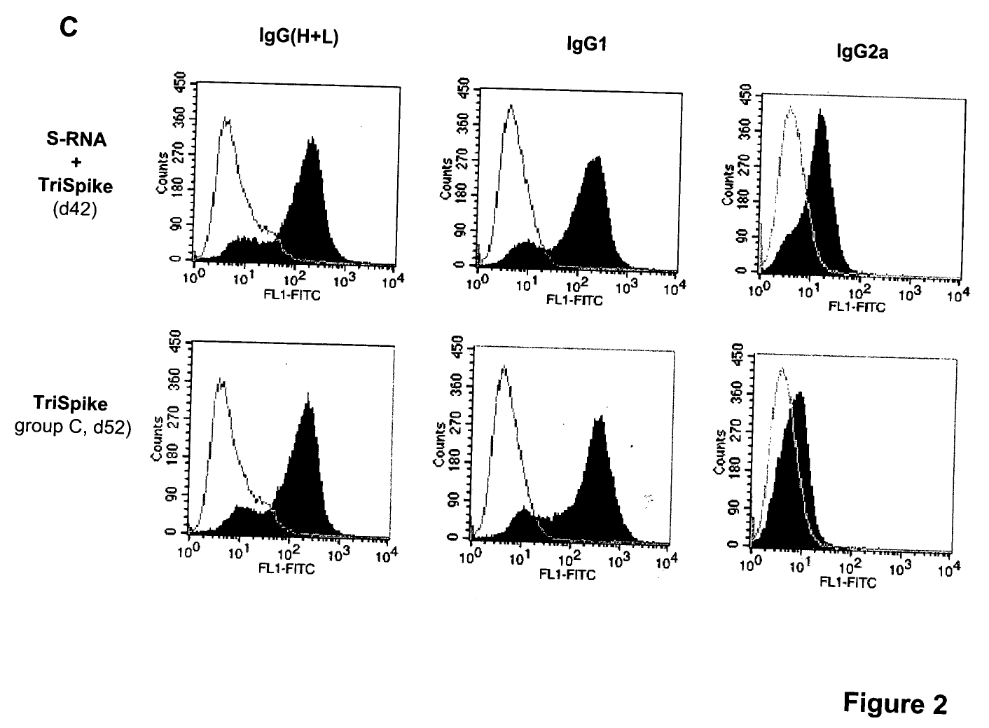

In order to assess the immunogenicity of TriSpike, two different immunization strategies were compared: TriSpike alone (2 or 3 immunizations in alum adjuvant) or in combination with an RNA vaccine, the defective replicating Semliki Forest Virus RNA coding for the S-protein (S-RNA). Sera collected at various time points were pooled and tested for reactivity with S-protein in Western Blot (FIG. 2 A, B) or at the surface of living cells by FACS (FIG. 2 C). Western blots were performed in conditions that partly dissociated trimers in order to allow the simultaneous detection of monomers, dimers and trimers of the S-protein (SDS and heat denaturation). A control protein, BAP-FLAG, was used to assess whether antibodies against the C-terminal FLAG tag were induced. Injection of S RNA did not induce detectable levels of anti-S antibodies (FIG. 2 A, d13). However, a single subsequent injection of TriSpike protein induced detectable levels of antibodies against S-protein (FIG. 2 A, d34) which could be further boosted by a second TriSpike injection (FIG. 2 A, d42). Sera were reactive against mono-, di- and trimers of S-proteins carrying either high-mannose or complex N-glycosylation and remained at high level until one month after the last boost (FIG. 2 A, d55, d76). Analysis of individual serum samples from d55 confirmed the homogeneity of the antibody response in individual mice (data not shown). It was then determined whether a comparable response could be induced by immunization with TriSpike alone using two or three injections (FIG. 2 B). A single injection with TriSpike results in a very weak anti-S antibody response at the limit of detection (FIG. 2 B, group A d13, group B d13). A second (FIG. 2 B, group A d52, group B d21) and third booster injection (FIG. 2 B, group B d52) strongly increased anti-S antibody levels. The 7-week time interval between first and second injection allowed for a stronger boost response (group A d52 versus group B d21). Analysis of individual serum samples from group B d21 confirmed the homogeneity of the antibody response in individual mice (data not shown). Neither S-RNA plus TriSpike nor TriSpike immunization alone induced antibodies directed against the FLAG peptide (FIG. 2 A, C, lower panels).

In order to analyze whether the strong anti-S response was also able to recognize non-denatured native S-protein at the surface of living cells, pooled sera shown in FIG. 2 A (d42) from S-RNA plus TriSpike immunized animals and pooled sera shown in FIG. 2 B (group B, d52) from TriSpike immunized animals were tested by FACScan. In both groups a strong reactivity of mouse IgG, predominantly of the IgG1 isotype, with plasma membrane-expressed S was observed (FIG. 2 C, left and middle panels). A subtle increase in induction of IgG2a isotype antibodies was observed when S-RNA plus TriSpike were used (FIG. 2 C, right panels). Altogether the immunogenicity results show that immunization with TriSpike protein alone induced a strong TH2 based response capable of detecting the native S-protein.

Recently it was shown that a UV-inactivated SARS-CoV induced a mixed TH1/TH2 response (Takasuka et al., 2004). In the FIPV model antibodies against the S-protein can induce antibody-mediated uptake and replication in macrophages leading to enhanced disease (antibody dependent enhancement or ADE) (Corapi et al., 1992; Hohdatsu et al., 1998). Interestingly, in vitro, IgG2a mAbs directed against the Feline Infectious Peritonitis Virus (FIPV) S-protein can enhance macrophage infection by 10 FIPV while IgG1 mAbs directed against the same epitope confer protection (Hohdatsu et al., 1994). The relevance of IgG isotype with respect to protection and potential ADE mediated immunopathology needs to be tested in a relevant challenge model which can reproduce SARS-CoV induced pathology or disease.

TriSpike Vaccinated Mice Sera Recognize SARS-CoV Infected Cells.

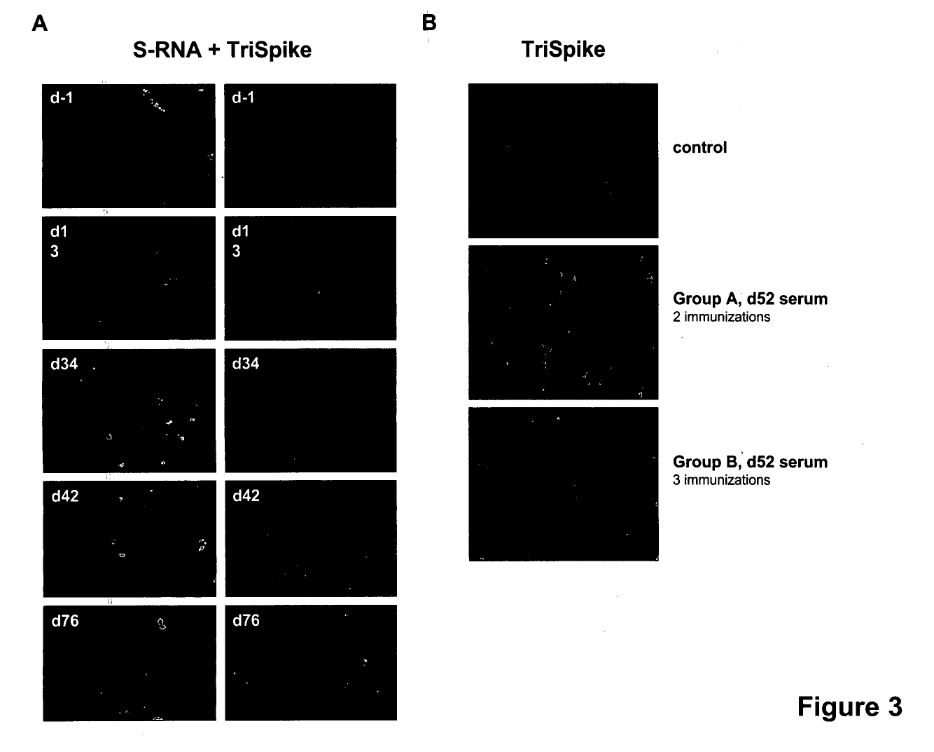

To further characterize the antibody response in vaccinated animals, immunofluorescence analyses was performed on SARS-CoV infected FRhk-4 or Vero cells (FIG. 3 A, B). Sera from both immunization groups effectively recognized SARS-CoV infected cells. In good correlation with data from Western Blot and FACS analyses (FIG. 2), a stronger recognition of SARS-CoV infected cells in sera of mice that have been boosted once or twice with TriSpike (FIG. 3 A, right panel) was observed. Altogether, these immunogenicity studies indicate that TriSpike has retained native antigenic properties allowing for the induction of antibodies against S-protein expressed by SARS-CoV.

High-Titer Neutralizing Antibodies in Sera from TriSpike Vaccinated Mice.

Next evaluated was the neutralizing activity of sera from TriSpike vaccinated mice. Serial dilutions of sera were tested for their neutralizing activity of cpe induced by SARS-CoV replication in FRhk-4 cells (neutralization of 100 TCID50). Injection of S RNA and a subsequent TriSpike protein booster did not induce nAb (Table 1). However, a second TriSpike booster injection induced high-titer nAb (1/2666). Without further immunization, neutralizing titers remained at 1/2400 at d55 (data not shown) and maintained at high levels until d116 (1/1200).

It was then determined whether a comparable high-titer neutralizing response could be induced by immunization with TriSpike alone (Table 1). No nAb were detected after a single TriSpike injection. A second booster injection induced nAb in group B at d21 (1/300) and group A at d52 (1/1200). Induction of nAb correlates with detection efficiency of S-protein by Western Blot (FIG. 2 B) and FACS (data not shown). Highest nAb titers were observed in group B mice at d52 after a third booster injection with TriSpike (1/6400). Without further immunization neutralizing titers remained at high levels until d104 in group A (1/666) and B (1/4266).

This invention clearly shows that purified trimeric S-protein can induce high-titer nAb when used alone, and therefore constitutes an important tool for the development of an efficacious vaccine against SARS-CoV. Peak neutralizing antibody titers are significantly higher than those obtained with sera from SARS patients tested with the same neutralization assay. nAb obtained in this invention appear to be significantly higher to titers obtained in other SARS-CoV vaccination studies (Bisht et al., 2004; Bukreyev et al., 2004; Gao et al., 2003; Subbarao et al., 2004; Takasuka et al., 2004; Yang et al., 2004b; Zeng et al., 2004; Zhang et al., 2004).

Neutralizing Sera Block Spike Binding to the ACE2 Receptor.

Next investigated was the mechanism of neutralization by analyzing the capacity of sera to block the interaction between immunopurified trimeric S-protein coated on sepharose beads with purified soluble ACE2, the SARS-CoV entry receptor (Li et al., 2003; Wang et al., 2004). FIG. 4 shows that sera from TriSpike immunized mice, but not from control animals, neutralized S-protein binding to the ACE2 receptors. These results suggest inhibition of receptor binding as a key immune response triggered by the TriSpike SARS vaccine. Recently, a human mAb from a nonimmune human antibody library was described which blocked association of S-protein with ACE2 (Sui et al., 2004). This invention shows that such antibodies can be induced by a purified protein vaccine with high efficiency. However, neutralization of receptor binding might not be the sole mechanism. Neutralization with antibodies against the putative S2 protein (Zhang et al., 2004) suggest that antibodies can also block post binding steps, e.g., conformational transitions of the S2 subunit required for membrane fusion.

Alternative approaches can be followed for the development of a vaccine against SARS based on nAb against the S-protein: whole inactivated vaccines (Takasuka et al., 2004), viral vectors, e.g., parainfluenzavirus (Buchholz et al., 2004; Bukreyev et al., 2004), MVA (Bisht et al., 2004), Adenovirus (Gao et al., 2003) and DNA vaccines (Yang et al., 2004b; Zeng et al., 2004). TriSpike alone was as efficient as a combination of a replicating viral vector and TriSpike in inducing nAb, leading to the conclusion that biochemically pure S-protein trimer is a viable vaccine for SARS.

In summary, viral receptor binding proteins are major targets of the host neutralizing antibody response. Here we present a recombinant native full-length S-protein trimer (TriSpike) of severe acute respiratory syndrome coronavirus (SARS-CoV) as vaccine candidate for the induction of neutralizing antibodies. TriSpike has native antigenicity and folding, as demonstrated by reactivity with IgG from SARS patient sera and binding to the ACE2 entry receptor. It induces a TH2-based antibody response in mice directed against denatured or native S-protein and SARS-CoV-infected cells. High titers of neutralizing antibody are detected in animals immunized and boosted with TriSpike. Titers drop within a month following the last immunization, but stabilize at a constant and high level. These titers are significantly higher than those observed in patients with SARS. Neutralizing sera block S-protein binding to the ACE2 receptor, suggesting inhibition of receptor binding as the major mechanism of neutralization in vaccinated animals. The results of the invention indicate that purified native trimeric S-protein is a key component of a safe and potent vaccine against SARS.

This invention will be described in greater detail in the following Examples.

### EXAMPLE 1

Spike (S) Protein Expression with Semliki Forest Virus Vectors (pSFV).

All DNA manipulations were handled according to standard procedures (Sambrook, 1989). Codon-optimized SARS-S DNA corresponding to sequence HKU-39849 was produced using GeneOptimizer™ Technology (GENEART, Regensburg, Germany). A FLAG sequence was included in frame at the 3′ end of SARS-S optimized cDNA. S-FLAG was sub-cloned into pSFV1 vector resulting in plasmid pSFV-S-FLAG. BHK-21 cells were directly transfected with in vitro transcribed S-RNA (Roche) or infected with S-FLAG-SFV pseudo-particles as previously described (Lozach et al., 2003).

### EXAMPLE 2

FLAG-Tag Immunoaffinity Purification and Analysis of Recombinant S-Protein.

The protein encoded by Sequence HKU-39849 is referred to herein as “trimeric S-protein (TriSpike)” of SARS-CoV.

The baby hamster kidney (BHK)-21 cell line was cultured at 37° C., 5% CO₂, in GMEM medium supplemented with 5% FCS, Hepes 20 mM, Tryptose-phosphate broth 10%, penicillin 100 U/ml and streptomycin 100 ug/ml. At 14 hours post-infection/transfection, BHK-21 cells were lysed (20 mM Tris-HCL 7.5, 150 mM NaCl, 2 mM EDTA, 1% Triton X-100) and incubated for 5 min on ice. The collected lysate was vortexed and incubated for another 15 min on ice prior to centrifugation at 13000 rpm for 15 min. Recombinant S-protein was immunoprecipitated from the supernatant using anti-FLAG M2 mAb-coated agarose beads (Sigma) overnight at 4° C. Subsequently, beads were washed three times with 1× washing buffer (Sigma) and recombinant S-protein was eluted with 3× FLAG peptide according to the supplier's instructions (Sigma). Eluted recombinant S-protein was concentrated and impurities below a molecular weight of 100 kDa removed with centrifugal filter devices (Amicon) according to the supplier's instructions. The quantity and quality of recombinant S-protein was assessed by SDS-PAGE and Western Blot using BAP-FLAG protein and microBSA methods as standards for protein quantification as previously described (Lozach et al., 2003; Staropoli et al., 2000). Briefly, protein samples were analyzed on 4-12% Bis-Tris SDS-PAGE gel (Invitrogen) under non-reducing conditions, except in experiments represented in FIG. 1 where different denaturing conditions were used as indicated. Proteins were transferred to PVDF membrane (Amersham Biosciences) and reacted with diluted mouse sera (1/500). After washing, the membrane was reacted with HRP-conjugated anti-mouse IgG (H+ L) (1/1000) (Zymed), followed by visualization of the bands on X-ray film (Kodak) using chemiluminescence (Amersham Biosciences). All steps were blocked with 3% normal goat serum (Zymed).

### EXAMPLE 3

Immunization with S-RNA and TriSpike.

In a first group of animals 6-8 weeks old, Balb/c mice (n=5 per group) were immunized intramuscularly (i.m.) with 25 μg of in vitro transcribed S-RNA on d0 followed by immunization with 60 μg of TriSpike protein in 1 mg of aluminium hydroxide gel (alum) on d14 and d35. Animals in the control group received empty SFV vector RNA at d0 and 1 mg of alum on the same days. A second set of 6-8 weeks old Balb/c mice (n=4 per group) were immunized with 60 μg of TriSpike protein in 1 mg of alum on d0 and d41 (group A) or d0, d14 and d41 (group B). Blood samples were collected by retro-orbital bleeding at indicated time points in accordance with local guidelines and sera were prepared and heat-inactivated.

### EXAMPLE 4

Flow Cytometry

Recombinant S-protein expressing BHK-21 cells and normal BHK-21 cells were detached with 5 mM EDTA and incubated for 45 min at 4° C. with the diluted mouse sera (1/100). After washing, the cells were fixed with 3.2% of PFA for 5 min at 4° C. After fixation, the cells were labeled with the fluorescein isothiocyanate-conjugated goat anti-mouse IgG (H+ L), rat anti-mouse IgG1 or IgG2a (1/100) (Zymed) for 30 min at 4° C. Finally, the cells were analyzed by flow cytometer (FACSCalibur, BD). All steps were blocked with 3% normal goat or rat serum (Zymed).

### EXAMPLE 5

Immunofluorescence of SARS-CoV Infected Cells

FRhk-4 cells grown on glass coverslips were infected with SARS-CoV, fixed with cold methanol/acetone 50:50 (v/v), and were incubated with diluted mouse sera (1/50) for 45 min at RT. After washing, the cells were labeled with Texas Red-conjugated goat antimouse IgG (H+ L) (1/100) for 30 min at RT and mounted (Sigma). Alternatively, SARS-CoV-infected VeroE6 cells (EUROIMMUN) were used. Slides were analyzed on a Zeiss Axiovert 200M microscope.

### EXAMPLE 6

Serum-Neutralization Assay

100 TCID50 of SARS-CoV (strain HKU-39849) were incubated for 2 hours at 37° C. with serial 2-fold dilutions of mouse sera in quadruplicate. Virus antibody mix was then added to FRhk-4 cells in 96-well plates and plates were incubated at 37° C. with microscopic examination for cytopathic effect (cpe) after a 4-day incubation. Neutralization titers were calculated by the Reed & Muench formula and are expressed as the reciprocal of the serum dilution which neutralized cpe in 50% of the wells (Reed and Muench, 1938). Mouse sera were heat-inactivated at 56° C. for 30 min.

### EXAMPLE 7

ACE2 Binding Assay

Recombinant S-protein tagged at its C-terminus end with a FLAG peptide or FLAG-BAP protein (Sigma) previously preadsorbed onto Anti-FLAG M2 affinity gel beads (Sigma) for 2 hours at 4° C. were incubated with soluble recombinant human ACE2 protein (R&D Systems) for 2 hours at 4° C. For inhibition of binding analysis, protein-coated beads were preincubated with sera for 1 hour at 4° C. before incubation with soluble ACE2. The beads were washed four times with lysis buffer (20 mM Tris-HCL 7.5, 150 mM NaCl, 2 mM EDTA, 1% Triton X-100). Precipitates were separated by SDS-PAGE followed by Western blotting with a goat anti-ACE2 ectodomain polyclonal antibody (R&D Systems). Immune complexes were detected with a mouse peroxydase-conjugated anti-goat IgG monoclonal antibody (1/1000) (Sigma), followed by visualization of the bands on X-ray film (Kodak) using chemiluminescence (Amersham Biosciences).

### REFERENCES

The entire disclosures of each of the following publications are relied upon and incorporated by reference herein.
1. Baudoux, P., Carrat, C., Besnardeau, L., Charley, B. and Laude, H. (1998) Coronavirus pseudoparticles formed with recombinant M and E proteins induce alpha interferon synthesis by leukocytes. J Virol, 72, 8636-8643.
2. Bisht, H., Roberts, A., Vogel, L., Bukreyev, A., Collins, P. L., Murphy, B. R., Subbarao,
3. K. and Moss, B. (2004) Severe acute respiratory syndrome coronavirus spike protein expressed by attenuated vaccinia virus protectively immunizes mice. Proc Natl Acad Sci USA, 101, 6641-6646.
4. Bosch, B. J., van der Zee, R., de Haan, C. A. and Rottier, P. J. (2003) The coronavirus spike protein is a class I virus fusion protein: structural and functional characterization of the fusion core complex. J Virol, 77, 8801-8811.
5. Buchholz, U. J., Bukreyev, A., Yang, L., Lamirande, E. W., Murphy, B. R., Subbarao,
6. K. and Collins, P. L. (2004) Contributions of the structural proteins of severe acute respiratory syndrome coronavirus to protective immunity. Proc Natl Acad Sci USA, 101, 9804-9809.
7. Bukreyev, A., Lamirande, E. W., Buchholz, U. J., Vogel, L. N., Elkins, W. R., St Claire, M., Murphy, B. R., Subbarao, K. and Collins, P. L. (2004) Mucosal immunisation of African green monkeys (Cercopithecus aethiops) with an attenuated parainfluenza virus expressing the SARS coronavirus spike protein for the prevention of SARS. Lancet, 363, 2122-2127.
8. Corapi, W. V., Olsen, C. W. and Scott, F. W. (1992) Monoclonal antibody analysis of neutralization and antibody-dependent enhancement of feline infectious peritonitis virus. J Virol, 66, 6695-6705.
9. Daniel, C. and Talbot, P. J. (1990) Protection from lethal coronavirus infection by affinity-purified spike glycoprotein of murine hepatitis virus, strain A59. Virology, 174, 87-94.
10. Gao, W., Tamin, A., Soloff, A., D'Aiuto, L., Nwanegbo, E., Robbins, P. D., Bellini, W. J., Barratt-Boyes, S. and Gambotto, A. (2003) Effects of a SARSassociated coronavirus vaccine in monkeys. Lancet, 362, 1895-1896.
11. Hohdatsu, T., Tokunaga, J. and Koyama, H. (1994) The role of IgG subclass of mouse monoclonal antibodies in antibody-dependent enhancement of feline infectious peritonitis virus infection of feline macrophages. Arch Virol, 139, 273-285.
12. Hohdatsu, T., Yamada, M., Tominaga, R., Makino, K., Kida, K. and Koyama, H. (1998) Antibody-dependent enhancement of feline infectious peritonitis virus infection in feline alveolar macrophages and human monocyte cell line U937 by serum of cats experimentally or naturally infected with feline coronavirus. J Vet Med Sci, 60, 49-55.
13. Ignjatovic, J. and Galli, L. (1994) The S1 glycoprotein but not the N or M proteins of avian infectious bronchitis virus induces protection in vaccinated chickens. Arch Virol, 138, 117-134.
14. Li, W., Moore, M. J., Vasilieva, N., Sui, J., Wong, S. K., Berne, M. A., Somasundaran,
15. M., Sullivan, J. L., Luzuriaga, K., Greenough, T. C., Choe, H. and Farzan, M. (2003) Angiotensin-converting enzyme 2 is a functional receptor for the SARS coronavirus. Nature, 426, 450-454.
16. Lozach, P. Y., Lortat-Jacob, H., de Lacroix de Lavalette, A., Staropoli, I., Foung, S., Amara, A., Houles, C., Fieschi, F., Schwartz, O., Virelizier, J. L., Arenzana-Seisdedos, F. and Altmeyer, R. (2003) DC-SIGN and L-SIGN are high affinity binding receptors for hepatitis C virus glycoprotein E2. J Biol Chem, 278, 20358-20366.
17. Peiris, J. S., Lai, S. T., Poon, L. L., Guan, Y., Yam, L. Y., Lim, W., Nicholls, J., Yee, W. K., Yan, W. W., Cheung, M. T., Cheng, V. C., Chan, K. H., Tsang, D. N., Yung, R. W., Ng, T. K. and Yuen, K. Y. (2003) Coronavirus as a possible cause of severe acute respiratory syndrome. Lancet, 361, 1319-1325.
18. Reed, L. J. and Muench, H. (1938) A simple method for estimating fifty percent endpoints. Am J Hyg, 27, 493-497.
19. Sambrook, J., Fritch, E. F. & Maniatis, T. (1989) Molecular Cloning: A Laboratory Manual (Cold Spring Harbor Lab. Press, Plainview, N.Y.).
20. Song, C. S., Lee, Y. J., Lee, C. W., Sung, H. W., Kim, J. H., Mo, I. P., Izumiya, Y., Jang, H. K. and Mikami, T. (1998) Induction of protective immunity in chickens vaccinated with infectious bronchitis virus S1 glycoprotein expressed by a recombinant baculovirus. J Gen Virol, 79 (Pt 4), 719-723.
21. Song, H. C., Seo, M. Y., Stadler, K., Yoo, B. J., Choo, Q. L., Coates, S. R., Uematsu, Y., Harada, T., Greer, C. E., Polo, J. M., Pileri, P., Eickmann, M., Rappuoli, R., Abrignani, S., Houghton, M. and Han, J. H. (2004) Synthesis and characterization of a native, oligomeric form of recombinant severe acute respiratory syndrome coronavirus spike glycoprotein. J Virol, 78, 10328-10335.
22. Staropoli, I., Chanel, C., Girard, M. and Altmeyer, R. (2000) Processing, stability, and receptor binding properties of oligomeric envelope glycoprotein from a primary HIV-1 isolate. J Biol Chem, 275, 35137-35145.
23. Subbarao, K., McAuliffe, J., Vogel, L., Fahle, G., Fischer, S., Tatti, K., Packard, M., Shieh, W. J., Zaki, S. and Murphy, B. (2004) Prior infection and passive transfer of neutralizing antibody prevent replication of severe acute respiratory syndrome coronavirus in the respiratory tract of mice. J Virol, 78, 3572-3577.
24. Sui, J., Li, W., Murakami, A., Tamin, A., Matthews, L. J., Wong, S. K., Moore, M. J., Tallarico, A. S., Olurinde, M., Choe, H., Anderson, L. J., Bellini, W. J., Farzan, M. and Marasco, W. A. (2004) Potent neutralization of severe acute respiratory syndrome (SARS) coronavirus by a human mAb to S1 protein that blocks receptor association. Proc Natl Acad Sci USA, 101, 2536-2541.
25. Takasuka, N., Fujii, H., Takahashi, Y., Kasai, M., Morikawa, S., Itamura, S., Ishii, K., Sakaguchi, M., Ohnishi, K., Ohshima, M., Hashimoto, S. I., Odagiri, T., Tashiro, M., Yoshikura, H., Takemori, T. and Tsunetsugu-Yokota, Y. (2004) A subcutaneously injected UV-inactivated SARS-coronavirus vaccine elicits systemic humoral immunity in mice. Int Immunol.
26. ter Meulen, J., Bakker, A. B., van den Brink, E. N., Weverling, G. J., Martina, B. E., Haagmans, B. L., Kuiken, T., de Kruif, J., Preiser, W., Spaan, W., Gelderblom, H. R., Goudsmit, J. and Osterhaus, A. D. (2004) Human monoclonal antibody as prophylaxis for SARS coronavirus infection in ferrets. Lancet, 363, 2139-2141.
27. Torres, J. M., Sanchez, C., Sune, C., Smerdou, C., Prevec, L., Graham, F. and Enjuanes, L. (1995) Induction of antibodies protecting against transmissible gastroenteritis coronavirus (TGEV) by recombinant adenovirus expressing TGEV spike protein. Virology, 213, 503-516.
28. Wang, P., Chen, J., Zheng, A., Nie, Y., Shi, X., Wang, W., Wang, G., Luo, M., Liu, H., Tan, L., Song, X., Wang, Z., Yin, X., Qu, X., Wang, X., Qing, T., Ding, M. and Deng, H. (2004) Expression cloning of functional receptor used by SARS coronavirus. Biochem Biophys Res Commun, 315, 439-444.
29. Yang, Z. Y., Huang, Y., Ganesh, L., Leung, K., Kong, W. P., Schwartz, O., Subbarao, K. and Nabel, G. J. (2004a) pH-dependent entry of severe acute respiratory syndrome coronavirus is mediated by the spike glycoprotein and enhanced by dendritic cell transfer through DC-SIGN. J Virol, 78, 5642-5650.
30. Yang, Z. Y., Kong, W. P., Huang, Y., Roberts, A., Murphy, B. R., Subbarao, K. and Nabel, G. J. (2004b) A DNA vaccine induces SARS coronavirus neutralization and protective immunity in mice. Nature, 428, 561-564.
31. Zeng, F., Chow, K. Y., Hon, C. C., Law, K. M., Yip, C. W., Chan, K. H., Peiris, J. S. and Leung, F. C. (2004) Characterization of humoral responses in mice immunized with plasmid DNAs encoding SARS-CoV spike gene fragments. Biochem Biophys Res Commun, 315, 1134-1139.
32. Zhang, H., Wang, G., Li, J., Nie, Y., Shi, X., Lian, G., Wang, W., Yin, X., Zhao, Y., Qu, X., Ding, M. and Deng, H. (2004) Identification of an antigenic determinant on the S2 domain of the severe acute respiratory syndrome coronavirus spike glycoprotein capable of inducing neutralizing antibodies. J. Virol, 78, 6938-6945.

What is claimed is:

1. A process for vaccinating humans in need thereof against SARS-CoV infection, which comprises the steps of:
a) administering to a human in need thereof, one or more times, a native or recombinant trimeric S-protein (TriSpike) of SARS-CoV inducing in vivo a neutralizing immune response against a SARS-CoV virus infection with an acceptable physiological carrier and/or an adjuvant.

2. The process according to claim 1, wherein the protein with an acceptable physiological carrier and/or an adjuvant is administered by intravenous route, intramuscular route, oral route, or mucosal route.

3. Purified antibodies that specifically bind to native or recombinant trimeric S-protein (TriSpike) of SARS-CoV.

4. Purified antibodies according to claim 3, wherein the antibodies are monoclonal antibodies.

5. An immunological complex comprising a trimeric S-protein (TriSpike) of SARS-CoV and an antibody that specifically recognizes said polypeptide.

6. A method for detecting infection by SARS-CoV, wherein the method comprises providing a composition comprising a biological material suspected of being infected with SARS-CoV, and assaying for the presence of trimeric S-protein (TriSpike) of SARS-CoV by reaction of the protein with an antibody as claimed in claim 3.

7. An in vitro diagnostic method for the detection of the presence or absence of trimeric S-protein (TriSpike) of SARS-CoV, wherein the method comprises contacting an antibody as claimed in claim 3 with a biological fluid for a time and under conditions sufficient for the protein in the biological fluid and the antibody to form an antigen-antibody complex, and detecting the formation of the complex.

8. The method as claimed in claim 7, which further comprises measuring the formation of the antigen-antibody complex.

9. The method as claimed in claim 7, wherein the formation of antigen-antibody complex is detected by immunoassay based on Western blot technique, ELISA, indirect immunofluorescence assay, or immunoprecipitation assay.

10. A diagnostic kit for the detection of the presence or absence of trimeric S-protein (TriSpike) of SARS-CoV, wherein the kit comprises an antibody as claimed in claim 3, and means for detecting the formation of immune complex between the protein and the antibody, wherein the means are present in an amount sufficient to perform said detection.

11. An immunogenic composition comprising at least one trimeric S-protein (TriSpike) in an amount sufficient to induce an immunogenic or protective response in vivo, and a pharmaceutically acceptable carrier therefore.

12. The immunogenic composition as claimed in claim 11, wherein said composition comprises a neutralizing amount of at least one trimeric S-protein (TriSpike).
